(19) United States
(12) Patent Application Publication
(10) Pub. No.: US 2022/0023623 A1
(43) Pub. Date: Jan. 27, 2022

(54) SONIC HEDGEHOG DELIVERY

(71) Applicant: Leonhardt Ventures LLC, Corona Del Mar, CA (US)

(72) Inventor: Howard J. Leonhardt, Corona Del Mar, CA (US)

(21) Appl. No.: 17/498,583

(22) Filed: Oct. 11, 2021

Related U.S. Application Data
(63) Continuation-in-part of application No. 16/847,351, filed on Apr. 13, 2020.
(60) Provisional application No. 62/988,345, filed on Mar. 11, 2020.
(60) Provisional application No. 62/834,309, filed on Apr. 15, 2019.

(51) Int. Cl. A61N 1/20; A61N 1/04

(52) U.S. Cl. CPC A61N 1/205; A61N 1/06; A61N 1/0452; A61N 1/0496

(57) ABSTRACT

Described is a low voltage, pulsed electrical stimulation device for controlling expression of sonic hedgehog (“Shh”), a useful protein, by tissues. Also described are methods of enhancing expression of sonic hedgehog in cells, particularly a method of stimulating the expression and/or release of Shh in a cell having a gene encoding Shh, wherein the method includes applying a bioelectric signal of less than 50 Hz (e.g., 5 Hz, 10 Hz, or 20 Hz) at a pulse width duration of, e.g., 1 ms, to the cell (e.g., directly, indirectly, or wirelessly), and wherein the amount of Shh expression enhanced by this bioelectric signal is greater than that seen with a prior art bioelectric muscle stimulation or bioelectric muscle contraction alone as may be determined by, e.g., by an analysis of the upregulation of mRNA level/GAPDH fold gene expression in the cell.

### CROSS-REFERENCE TO RELATED APPLICATIONS

This application is a continuation-in-part of U.S. patent application Ser. No. 16/847,351, filed Apr. 13, 2020, which claims the benefit under 35 U.S.C. § 119(e) of U.S. Provisional Patent Application Ser. No. 62/834,309, filed Apr. 15, 2019, and U.S. Provisional Patent Application Ser. No. 62/988,345, filed Mar. 11, 2020, the disclosures of each of which are incorporated herein in their entirety by this reference.

### TECHNICAL FIELD

The application relates generally to the field of medical devices and associated methods of treatment, and more specifically to methods of treatment involving the precise bioelectrical stimulation of a subject's tissue, optionally augmented with the administration of a composition comprising, among other things, stem cells and nutrients, useful to increase the expression and/or release of sonic hedgehog to stimulate and treat the subject, the subject's tissue(s), the subject's organ(s), and/or the subject's cells. More specifically, the application relates to a device, programmed bioelectric signaling sequences, and associated methods for the controlled expression of secreted Sonic hedgehog (“Shh”), a known powerful anti-aging and regeneration promoting protein, via precise bioelectrical signaling sequences. In particular, described is a system, method, and associated components for treating erectile dysfunction (ED).

### BACKGROUND

Sonic hedgehog (Shh), a member of the hedgehog (Hh) family, was originally recognized as a morphogen possessing critical characters for organ development during embryogenesis. Shh has also emerged as an important modulator in adult tissues through different mechanisms such as anti-oxidation, anti-inflammation, and autophagy.

### BRIEF SUMMARY

Described herein is a bioelectric stimulator particularly configured to modulate (e.g., upregulate and/or downregulate) expression and/or release of Sonic hedgehog in cellular tissue.

Also described is a method for treating or regenerating a tissue in a subject (e.g., in an animal, mammal or human), the tissue selected from the group consisting of muscle, heart, eye, liver, dental tissue and teeth, bone, adrenal gland, pancreas, brain, skin, and lung, the method comprising: applying a bioelectric signal to the tissue, which the bioelectric signal regulates (e.g., upregulates or downregulates) the expression and/or release of secreted Sonic hedgehog.

One such precise bioelectric signal to upregulate Shh is a biphasic pulse at from 50 Hz to 100 Hz. The current delivered may be, for example, (at the cellular level of the cells being stimulated), from 100 μA to 500 μA.

Another precise bioelectric signal to upregulate Shh has a frequency/pulse width duration of 5 Hz/1 ms (e.g., as measured at the level of the cells being stimulated for expression of SHH, 100 mV, biphasic, 5 Hz, 1 ms, voltage-driven system).

Another precise bioelectric signal to upregulate Shh has a frequency/pulse width duration of 10 Hz/1 ms (e.g., as measured at the level of the cells being stimulated for expression of SHH, 100 mV, biphasic, 1 ms, voltage-driven system).

Another precise bioelectric signal to upregulate Shh has a frequency/pulse width duration of 20 Hz/1 ms (e.g., as measured at the level of the cells being stimulated for expression of SHH, 100 mV, biphasic, 1 ms, voltage-driven system).

Another precise bioelectric signal to upregulate Shh has a frequency/pulse width duration of 50 Hz/1 ms (e.g., as measured at the level of the cells being stimulated for expression of SHH, 100 mV, biphasic, 1 ms, voltage-driven system).

Another precise bioelectric signal to upregulate Shh has a frequency/pulse width duration of 100 Hz/1 ms (e.g., as measured at the level of the cells being stimulated for expression of SHH, 100 mV, biphasic, 1 ms, voltage-driven system).

Further described is a method for treating a subject (e.g., in an animal, mammal or human), wherein the subject is in need of wound healing, hair regrowth and regeneration, and/or treatment of erectile dysfunction, the method comprising: applying a bioelectric signal to the subject, which signal regulates (e.g., upregulates or downregulates) the expression wherein the bioelectric signal secreted Sonic hedgehog.

In certain embodiments, described is a method of treating a subject for erectile dysfunction (ED), the method comprising: applying an electrode to tissue at or near the subject's groin area so as to provide a bioelectric signal that increases the expression of Sonic hedgehog (SHH) in the tissue, wherein the bioelectric signal as measured at the cellular level of the tissue comprises: a biphasic pulse with/at a frequency/amplitude range of 40 Hz to 100 Hz at greater than or equal to 100 μA, or a biphasic pulse at from 5 Hz to 20 Hz at greater than or equal to 100 μA.

In such embodiments, the electrode is preferably a gel electrode. Preferably, the gel electrode has been modified or manufactured to comprise a gel comprising SHH. In certain embodiments, the gel electrode gel further comprises glyceryl trinitrate.

In certain described methods, the method further comprises applying an electrode gel to the electrode or to the groin area before application of the electrode to the groin area, wherein the electrode gel comprises SHH. In certain embodiments, the electrode gel further comprises glyceryl trinitrate.

In certain described methods, the method further comprises applying an electrode gel to the electrode or to the groin area before application of the electrode to the groin area, wherein the electrode gel comprises glyceryl trinitrate.

In certain embodiments, the voltage and the current are increased until the (e.g., human) subject feels a slight tingling at the stimulation area.

One such precise bioelectric signal to upregulate Shh is a biphasic pulse at from 50 Hz to 100 Hz at from 100 μA to 500 μA (e.g., as measured at the level of the cells being stimulated). Another precise bioelectric signal to upregulate Shh has a frequency/pulse width duration of 5 Hz/1 ms (e.g., as measured at the level of the cells being stimulated for expression of SHH, 100 mV, biphasic, 1 ms, voltage-driven system). Another precise bioelectric signal to upregulate Shh has a frequency/pulse width duration of 10 Hz/1 ms (e.g., as measured at the level of the cells being stimulated for expression of SHH, 100 mV, biphasic, 1 ms, voltage-driven system). Another precise bioelectric signal to upregulate Shh has a frequency/pulse width duration of 20 Hz/1 ms (e.g., as measured at the level of the cells being stimulated for expression of SHH, 100 mV, biphasic, 1 ms, voltage-driven system). Another precise bioelectric signal to upregulate Shh has a frequency/pulse width duration of 50 Hz/1 ms (e.g., as measured at the level of the cells being stimulated for expression of SHH, 100 mV, biphasic, 1 ms, voltage-driven system). Another precise bioelectric signal to upregulate Shh has a frequency/pulse width duration of 100 Hz/1 ms (e.g., as measured at the level of the cells being stimulated for expression of SHH, 100 mV, biphasic, 1 ms, voltage-driven system).

In certain embodiments, the bioelectric stimulator is further configured to activate expression and/or release of another protein, such as stromal cell-derived factor 1 (“SDF-1”), insulin-like growth factor 1 (“IGF-1”), platelet-derived growth factor (“PDGF”), follistatin, tropoelastin, and any combination thereof.

Also described is a bioelectric stimulator including: a power source (e.g., battery, capacitor, AC, or other suitable source of electricity), and means for delivering an electrical signal to a subject's tissue (e.g., via electrode(s) or wirelessly). The bioelectric stimulator utilizes the electrical signal to precisely control Shh expression in the tissue on demand.

In certain cases, the bioelectric stimulator is programmed to produce a bioelectric signal that stimulates target tissue to express and/or release Sonic hedgehog polypeptide by the target tissue by utilizing a biphasic pulse at from 50 Hz to 100 Hz at from 100 μA to 500 μA for a period of time from about 15 minutes to about an hour of stimulation. In certain embodiments, stimulation times of thirty (30) minutes work well, especially with up regulation. For typical treatments, this may be applied to the subject's tissue and repeated daily, or 1, 2, 3, 4, 5, 6, 7, or more times a week.

The amount of Sonic hedgehog expression enhanced by the herein described system is greater than with that seen with prior art bioelectric muscle stimulation or from muscle contraction alone.

In certain embodiments, described is a method of stimulating the expression and/or release of Shh in a cell having a gene encoding Shh, wherein the method includes applying a bioelectric signal of less than 50 Hz (e.g., 5 Hz, 10 Hz, 20 Hz, or 40 Hz) at a pulse width duration of, e.g., 1 ms, to the cell (e.g., directly or wirelessly), and wherein the amount of Shh expression enhanced by this bioelectric signal is greater than that seen with a prior art bioelectric muscle stimulation or bioelectric muscle contraction alone (e.g., “ESTIM”) as may be determined by, e.g., by an analysis of the upregulation of mRNA level/GAPDH fold gene expression in the cell.

Using the bioelectric stimulator as described herein, in certain experiments, Sonic hedgehog expression in cells has been upregulated by up to 250%. In other experiments, the increase was at an average of 219% (range 127% to 847%) over baseline.

The upregulation of expression of SHH leads to an extended and enhanced release of SHH by the treated cells.

In certain cases, a method of using the bioelectric stimulator to stimulate tissue of a subject includes connecting (directly or wirelessly) the bioelectric stimulator to the target tissue or cells of the subject. The target tissue may be selected from, e.g., the group consisting of muscle, heart, eye, liver, pancreas, brain, skin, and lung.

In certain cases, the subject is interested in body building and the stimulation is preferably at the muscle level.

In certain cases, the subject has been diagnosed as is in need of wound healing, hair regeneration, and/or treatment of erectile dysfunction.

A preferred system includes: a bioelectric stimulator that controls/stimulates the release/production of Sonic hedgehog by a target cell or tissue. The stimulator may be associated with (e.g., connected to) the organ or tissue to be treated with a pacing infusion lead (available from Nanoscribe of Eggenstein-Leopoldshafen, Germany) or wirelessly. In certain cases, the interface with the subject's tissue may be by a conductive soft wrap.

The stimulator can be designed to externally deliver all regeneration promoting signals wirelessly to the subject's organ(s), tissue(s), and/or cells. In certain embodiments, a micro infusion pump may be included in the system to deliver other supportive substances (such as stem cells) in greater volume more quickly.

While not intending to be bound by theory, the described system utilizes precise bioelectric signaling sequences that appear to communicate with the cells, cell membranes, and DNA of the subject to cause the cells to produce high volumes of the Sonic hedgehog protein. Potential indications include muscle regeneration and treatment, heart treatment and regeneration, eye treatment and regeneration, liver treatment and regeneration, pancreas treatment and regeneration, brain (and other nervous tissue) protection, treatment, and regeneration, wound healing, hair regeneration, skin treatment and regeneration, lung treatment and regeneration, and treatment of erectile dysfunction. A goal is to use the described technology to help patients keep their own organs, regenerated back to full health, instead of getting artificial, different species, or donor transplant implants

### BRIEF DESCRIPTION OF THE DRAWINGS

FIG. 1 depicts a programmed bioelectric stimulator for delivery to a subject connected to multiple soft conductive electrode pads.

FIG. 2 depicts a programmed bioelectric stimulator as described herein.

FIG. 3 depicts a conductive soft wrap for use with the system.

FIG. 4 depicts a programmed bioelectric stimulator depicted alongside a pen.

FIG. 5 depicts a kit assembly for a system described herein.

### DETAILED DESCRIPTION

In certain embodiments, described is a bandage wrap that is applied to the affected region. A micro-stimulator may be located conveniently in the bandage wrap and is utilized to distribute specific bioelectric signals to the affected tissue and nerves that regulate various protein expressions for stem cell homing, stem cell proliferation, stem cell differentiation, blood vessel formation, blood circulation improvement, muscle function repair, and DNA repair.

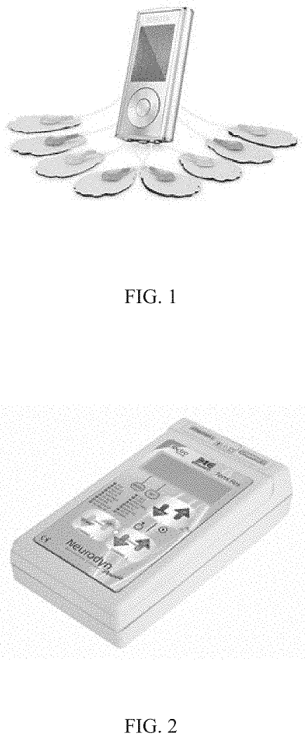

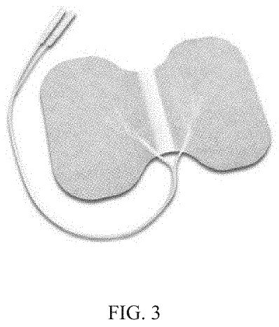

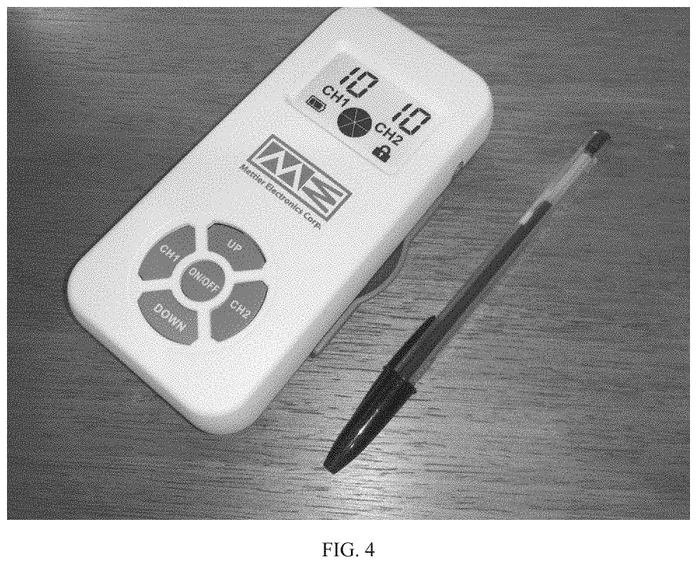

Referring now to FIG. 1, depicted is a stimulator for use in treating a human. The depicted device is about the size of a pen (FIG. 4) and is programmable.

Preferably, the system utilizes a bioelectric stimulator programmed to control expression and/or release of secrete Sonic hedgehog. As described in Su, Y. et al. High frequency stimulation induces sonic hedgehog release from hippocampal neurons. Sci. Rep. 7, 43865; doi: 10.1038/srep43865 (2017), the contents of which are incorporated herein by this reference, high, but not low, frequency stimulation induces Shh release from neurons, but not from astrocytes.

A bench top stimulator (e.g., a Mettler Model 240 Stimulator from Mettler Electronics of Anaheim, Calif., US) may be pre-programmed with the bioelectric signaling sequence(s) for controlling the expression and/or release of Shh.

In some embodiments (e.g., for the regulation of the production of new neurons or new bone growth), the bioelectric signaling can further be used to modulate (e.g., upregulate) by the subject's cells the production of other molecules in addition to Shh and/or the recruitment of stem cells. See, e.g., US 20180064935 A1 to Leonhardt et al. (Mar. 8, 2018), the contents of which are incorporated herein by this reference.

For treating and/or regenerating heart utilizing Shh, see, e.g., Kawagishi et al., “Sonic hedgehog signaling regulates the mammalian cardiac regenerative response”, J Mol Cell Cardiol. 2018 October; 123:180-184, doi: 10.1016/j.yjmcc.2018.09.005. Epub 2018 Sep. 17, Levin et al. “Endogenous Bioelectric Signaling Networks: Exploiting Voltage Gradients for Control of Growth and Form” Annual Review of Biomedical Engineering 19(1):353-387, June 2017, Wang et al. “Epicardial regeneration is guided by cardiac outflow tract and Hedgehog signalling” Nature 522(7555), May 2015, and Dunaeva et al. “Hh signaling in regeneration of the ischemic heart” Cell Mol Life Sci. 2017; 74(19): 3481-3490, the contents of each of which are incorporated herein by this reference.

For treating and/or regenerating liver utilizing Shh, see, e.g., Ochoa et al., “Hedgehog signaling is critical for normal liver regeneration after partial hepatectomy in mice”, Hepatology. 2010 May; 51(5): 1712-1723. doi: 10.1002/hep.23525, the contents of which are incorporated herein by this reference.

In certain embodiments, the person treated with the bioelectric stimulator is suffering from gastrointestinal distress or a less than healthy biome, which distress SHH helps to alleviate. See, Fu et al. “Sonic hedgehog regulates the proliferation, differentiation, and migration of enteric neural crest cells in gut” J Cell Biol (2004) 166 (5): 673-684, the contents of which is incorporated by this reference.

For treating and/or regenerating pancreas utilizing Shh, see, e.g., Fendrich et al., “Hedgehog Signaling Is Required for Effective Regeneration of Exocrine Pancreas”, Gastroenterology. 2008 August; 135(2): 621-631 and Frendrich et al. “Hedgehog Signaling Is Required for Effective Regeneration of Exocrine Pancreas” Gastroenterology 135(2):621-31 May 2008, the contents of each of which are incorporated herein by this reference.

For protecting, treating, and regenerating the brain, neurons and nervous tissue utilizing Shh (including recovery from stroke), see, e.g., Shang-Der Chen et al., “Emerging Roles of Sonic Hedgehog in Adult Neurological Diseases: Neurogenesis and Beyond”, Int J Mol Sci. 2018 August; 19(8): 2423, Chechneva et al “Empowering sonic hedgehog to rescue brain cells after ischemic stroke” Neural Regen Res. 2015 March; 10(3): 360-362, doi: 10.4103/1673-5374.153677, Wu et al. “Neuroprotective Effect of Upregulated Sonic Hedgehog in Retinal Ganglion Cells Following Chronic Ocular Hypertension” IOVS June 2010 Volume 51, Issue 6, and Chen et al. “Administration of sonic hedgehog protein induces angiogenesis and has therapeutic effects after stroke in rats” Neuroscience. 2017 Jun. 3; 352:285-295, doi: 10.1016/j.neuroscience.2017.03.054. Epub 2017 Apr. 5, the contents of each of which are incorporated herein by this reference. A preferred cell for treating and regenerating the brain is the neuron. See, also, Giarretta et al. “The Hedgehog Signaling Pathway in the Ischemic Heart, Brain, and Skeletal Muscle.” Preprints 2018, 2018080027 (doi: 10.20944/preprints201808.0027.v1), the contents of which are incorporated herein by this reference.

For treating and/or regenerating eye utilizing Shh, see, e.g., the herein incorporated Wu et al. (2010), Stenkamp et al., “Optimizing Retinal Regeneration: A Role for Sonic Hedgehog”, Investigative Ophthalmology & Visual Science April 2010, Vol. 51(13), 4309 and Spence et al. “The hedgehog pathway is a modulator of retina regeneration” Development, 2004 September; 131(18):4607-21, the contents of each of which are incorporated herein by this reference.

For treating and/or regenerating teeth and tooth roots utilizing Shh, see, e.g., Dassule et al. “Sonic hedgehog regulates growth and morphogenesis of the tooth” Development, 2000 127: 4775-4785 and Nakamoti et al. “Sonic hedgehog signaling is important in tooth root development”, J Dent Res. 2006 May; 85(5):427-31, the contents of each of which are incorporated herein by this reference.

For treating and/or regenerating bone utilizing Shh, see, e.g., Huang et al., “Overexpressing sonic hedgehog peptide restores periosteal bone formation in a murine bone allograft transplantation model” Mol Ther. 2014 February; 22(2):430-439. doi: 10.1038/mt.2013.222. Epub 2013 Oct. 3, K. Song “Enhanced bone regeneration with sequential delivery of basic fibroblast growth factor and sonic hedgehog” Injury 2011 August; 42(8):796-802. doi: 10.1016/j.injury.2011.02.003. Epub (2011 Mar. 1), M. Ueda, “Bone regeneration using osteogenic stem cell and sonic hedgehog gene”, J-Stage 2003 Volume 23 Issue 5 Pages 262-268, and Matsumoto et al. “Expression and Role of Sonic Hedgehog in the Process of Fracture Healing with Aging”, In Vivo March-April 2016, 30(2): 99-105, the contents of each of which are incorporated herein by this reference.

For wound healing utilizing Shh, see, e.g., the previously incorporated Chen et al. (2017), Han et al., “Multivalent Conjugates of Sonic Hedgehog Accelerate Diabetic Wound Healing”, Tissue Eng Part A. 2015 September; 21(17-18): 2366-78, doi: 10.1089/ten.TEA.2014.0281, Luo et al. “Sonic hedgehog improves delayed wound healing via enhancing cutaneous nitric oxide function in diabetes” Am J Physiol Endocrinol Metab. 2009 August; 297(2): E525-E531, and Wang et al. “Sonic Hedgehog Accelerates Wound Healing Via Enhancing Cutaneous Nitric Oxide Function in Diabetes” Advances in Wound Care: Volume 2, (May 9, 2011), the contents of each of which are incorporated herein by this reference.

In certain embodiments, the person treated with the bioelectric stimulator is suffering from or at risk of suffering from a disease associated with decreased nitric oxide production, which SHH helps alleviate. See, e.g., Marrachelli et al. “Sonic hedgehog carried by microparticles corrects angiotensin II-induced hypertension and endothelial dysfunction in mice.” PLoS One. 2013 Aug. 16; 8(8):e72861. doi: 10.1371/journal.pone.0072861, the contents of which is incorporated by this reference.

In certain embodiments, the person treated with the bioelectric stimulator is suffering from epithelial wounds, sensory nerve damage, and defects in the cornea, particularly those associated with diabetes, which distress SHH helps to alleviate. See, Zhang et al. “Role of VIP and Sonic Hedgehog Signaling Pathways in Mediating Epithelial Wound Healing, Sensory Nerve Regeneration, and Their Defects in Diabetic Corneas” Diabetes 2020 July; 69(7): 1549-1561; Park et al. “Sonic hedgehog intradermal gene therapy using a biodegradable poly(β-amino esters) nanoparticle to enhance wound healing” Biomaterials 33(35):9148-9156 (2012); doi.org/10.1016/j.biomaterials.2012.09.005, the contents of which each of which are incorporated herein by reference.

In certain embodiments, the subject is in need of nerve regeneration, and is treated with the bioelectric stimulator to upregulate expression of SHH and is also preferably either administered Insulin-like growth factor 1 (IGF-1) and/or LIM or is administered a bioelectric signal to upregulate expression of, e.g., IGF-1 (e.g., within 15%, a frequency of about 22 Hz, see, e.g., U.S. Pat. No. 10,960,206 to Leonhardt et al., the contents of which is incorporated herein by this reference).

In certain embodiments, the person treated with the bioelectric stimulator is suffering from or at risk of suffering from Parkinson's disease. In Parkinson's, the patient's brain loses the neurons that produce the brain molecule dopamine. Although treatments exist to replace dopamine with a molecule called L-dopa, doing so also tends to cause involuntary tremors known as L-dopa induced dyskinesia. Combining L-dopa treatment with agonists that increase the activity of Sonic hedgehog prevents those tremors. Malave, L., Zuelke, D. R., Uribe-Cano, S. et al. “Dopaminergic co-transmission with sonic hedgehog inhibits abnormal involuntary movements in models of Parkinson's disease and L-Dopa induced dyskinesia.” Commun Blol 4, 1071 (August 2021); doi.org/10.1038/s42003-021-02567-3, the contents of which is incorporated by this reference.

In certain embodiments, the person treated with the bioelectric stimulator is suffering from kidney damage, which distress SHH helps to alleviate. See, e.g., Zhou, Dong et al. “Sonic hedgehog signaling in kidney fibrosis: a master communicator.” Science China. Life sciences vol. 59, 9 (2016): 920-9. doi:10.1007/s11427-016-0020-y, the contents of which is incorporated by this reference.

For hair regeneration utilizing Shh, see, e.g., N. Lavars, “Scientists fire up sonic hedgehog gene to spawn new hair regrowth possibilities”, New Atlas (Nov. 28, 2018), Lim et al. “Hedgehog stimulates hair follicle neogenesis by creating inductive dermis during murine skin wound healing” Nature Communications Vol. 9, Article number: 4903 (2018), T. Newman “Could sonic hedgehog be the answer to hair loss?” Medical News Today (Dec. 1, 2018), St-Jacques et al. “Sonic hedgehog signaling is essential for hair development” Curr Biol. 1998 Sep. 24; 8(19): 1058-68, Abe et al. “Roles of the Hedgehog Signaling Pathway in Epidermal and Hair Follicle Development, Homeostasis, and Cancer” J Dev Biol. 2017 December; 5(4): 12, the contents of each of which are incorporated herein by this reference. A preferred treatment tissue for regenerating hair is the follicle.

In certain embodiments, the person treated with the bioelectric stimulator to upregulate SHH expression would benefit from the resultant modulation of stem cells in the subject's hair follicles. A variety of molecules are involved in the networks that critically regulate the fate of hair follicle stem cells, such as factors in hair follicle growth and development (e.g., in the Sonic hedgehog pathway), and that suppress apoptotic cues (the apoptosis pathway). Hu et al. “A systematic summary of survival and death signalling during the life of hair follicle stem cells.” Stem Cell Res Ther. 2021 Aug. 11; 12(1):453, the contents of which is incorporated by this reference.

For treating and/or regenerating skin utilizing Shh, see, e.g., the previously incorporated Abe et al. (2017), Suh et al. “Sonic hedgehog increases the skin wound-healing ability of mouse embryonic stem cells through the microRNA 200 family” Br J Pharmacol. 2015 February; 172(3): 815-828, and J. Tae, “Researchers discover new skin regeneration mechanism”, Yale News Dec. 4, 2018, the contents of which are incorporated herein by this reference.

For treating erectile dysfunction utilizing Shh, see, e.g., Choe et al., “Sonic hedgehog delivery from self-assembled nanofiber hydrogels reduces the fibrotic response in models of erectile dysfunction”, Acta Biomaterialia Volume 32, 1 Mar. 2016, pp. 89-99, Bond et al. “Sonic Hedgehog Regulates Brain-Derived Neurotrophic Factor in Normal and Regenerating Cavernous Nerves”. J Sex Med 2013; 10(3):730-737; Podlasek et al. “Sonic hedgehog, the penis and erectile dysfunction: a review of sonic hedgehog signaling in the penis” Curr Pharm Des. 2005; 11(31):4011-27; and Dobbs et al. “019 Sonic Hedgehog Promotes Cavernous Nerve Regeneration by Inducing Cavernous Nerve Sprouting and Sprouting Potential is Reduced with Age” J Sex Med February 2018 Volume 15, Issue 2, Supplement 1, Page S10, the contents of each of which are incorporated herein by this reference.

In certain embodiments, the person treated with the bioelectric stimulator is suffering from erectile dysfunction, which distress SHH helps to alleviate. See, e.g., Angeloni et al. “Regeneration of the cavernous nerve by sonic hedgehog using aligned peptide amphiphile nanofibers.” Biomaterials. 2011; 32:1091-1101; Bond et al. “Peptide amphiphile nanofiber delivery of sonic hedgehog protein to reduce smooth muscle apoptosis in the penis after cavernous nerve resection.” J Sex Med. 2011 January; 8(1):78-89; Choe et al. “Optimization of Sonic Hedgehog Delivery to the Penis from Self-Assembling Nanofiber Hydrogels to Preserve Penile Morphology after Cavernous Nerve Injury.” Nanomedicine. 2019 August; 20: 102033; Choe et al. “Sonic hedgehog delivery from self-assembled nanofiber hydrogels reduces the fibrotic response in models of erectile dysfunction.” Acta Biomater. 2016 Mar. 1; 32:89-99; Dobbs et al. “Sonic hedgehog regulation of cavernous nerve regeneration and neurite formation in aged pelvic plexus.” Exp Neurol. 2019 February; 312:10-19; Dobbs et al. “Peptide amphiphile delivery of sonic hedgehog protein promotes neurite formation in penile projecting neurons.” Nanomedicine. 2018 October; 14(7):2087-2094; Martin et al. “Peptide amphiphile nanofiber hydrogel delivery of Sonic hedgehog protein to the penis and cavernous nerve suppresses intrinsic and extrinsic apoptotic signaling mechanisms, which are an underlying cause of erectile dysfunction.” Nanomedicine. 2021 October; 37:102444; M. Paul “Protein Could Heal Erectile Dysfunction after Surgery” Northwestern Now (Jun. 8, 2010); northwestern.edu/newscenter/stories/2010/06/sonic.html; Podlasek C A. “Sonic hedgehog, apoptosis, and the penis.” J Sex Med. 2009 March; 6 Suppl 3(Suppl 3):334-9; Podlasek et al. “Sonic hedgehog, the penis and erectile dysfunction: a review of sonic hedgehog signaling in the penis.” Curr Pharm Des. 2005; 11(31):4011-27, the contents of which each of which are incorporated herein by reference.

In certain embodiments, particularly those for treating erectile dysfunction, the electrodes are gel electrodes wherein the gel thereof comprises Sonic hedgehog for delivery to the subject. Gel electrodes are well known in the art and are devices that use a gel to carry an electric current from the skin to an instrument. A sticky patch may hold the gel electrode on the skin so that the electrical activity of the heart or brain can be measured.

It has been shown that Sonic hedgehog treatment of the penis and cavernous nerve (“CN”) by peptide amphiphile nanofiber hydrogel reduces apoptosis, preserves smooth muscle, and suppresses collagen induction that occurs in response to CN crush injury. Choe et al. “Optimization of Sonic Hedgehog Delivery to the Penis from Self-Assembling Nanofiber Hydrogels to Preserve Penile Morphology after Cavernous Nerve Injury.” Nanomedicine. 2019 August; 20, U.S. Pat. No. 10,342,968 to Hasui et al. (Jul. 9, 2019) for “Electrode pad used for iontophoresis treatment”, the contents of each of which are incorporated by this reference.

Recombinant human Sonic hedgehog is commercially available (see, e.g., abcam ab123773 and BioVision, Inc. Catalog #4010). Dosages for delivery vary (e.g., from about 10 to 20 μg/kg subject body weight) and may be optimized as per the incorporated journal article to Chloe et al. 2019 infra.

Sonic hedgehog gel as self-assembling peptide amphiphiles may be incorporated into a gel electrode such as the herein described Mettler gel tape electrodes for incorporation into the system hereof. The use of self-assembling peptide amphiphiles (PA) as biological delivery vehicles, to prevent ED-related smooth muscle apoptosis in the penis is described in Bond et al. “Peptide amphiphile nanofiber delivery of sonic hedgehog protein to reduce smooth muscle apoptosis in the penis after cavernous nerve resection.” J Sex Med. 2011; 8:78-89; and Angeloni et al. “Regeneration of the cavernous nerve by sonic hedgehog using aligned peptide amphiphile nanofibers.” Biomaterials. 2011; 32: 1091-1101, the contents of each of which are incorporated by this reference. These PA systems deliver SHH delivery to the luminal surfaces of the corpora cavernosa (via in situ gelation), and to the injured cavernous nerve from a manipulable supramolecular cable (via monodomain aligned nanofibers). See, Choe et al. “Sonic hedgehog delivery from self-assembled nanofiber hydrogels reduces the fibrotic response in models of erectile dysfunction.” Acta Biomater. 2016 Mar. 1; 32:89-99, the contents of each of which are incorporated by this reference.

In certain embodiments, the PA systems for delivery of SHH are combined with the herein described bioelectric signal to upregulate expression of SHH.

In certain embodiments, the combination PA SHH delivery and bioelectric signal treatment is further supplemented with, e.g., topically-applied glyceryl trinitrate (GTN) for treating, for example, erectile dysfunction (ED) (e.g., topical application to the penis of MED2005, a topical GTN formulation using DermaSys® technology, which entails 300 mg of a 0.2% glyceryl trinitrate gel, for use at least 4 times during a 4 week period). See, e.g., Davis and Reisman “Development of a novel topical formulation of glyceryl trinitrate for the treatment of erectile dysfunction” Int Impoi Res 32, 569-577 (2020) the contents of each of which are incorporated by this reference. GIN is commercially available.

The GTN may be incorporated into the gel electrodes for delivery to the groin area (e.g., penis). The GTN may alternatively (or additionally) be incorporated into an electrode gel, which is applied either to the gel electrode surface contacting the patient's skin or area to which the electrode is to be applied. Concentrations of GTN vary from e.g., 0.1% to about 1%. See, e.g., Scholefield et al. “A dose finding study with 0.1%, 0.2%, and 0.4% glyceryl trinitrate ointment in patients with chronic anal fissures.” Gut vol. 52, 2 (2003): 264-9. doi:10.1136/gut.52.2.264, the contents of which are incorporated by this reference.

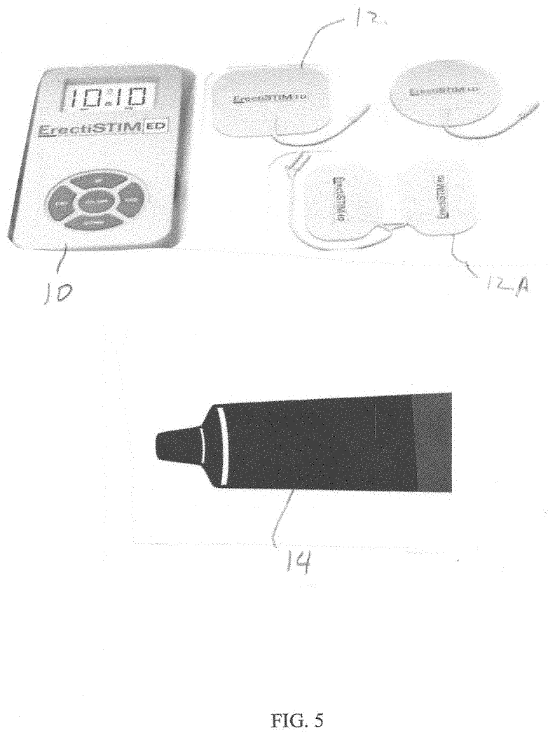

FIG. 5 depicts a kit assembly for a system which is particularly useful for treating ED. The kit includes a bioelectric stimulator 10 programmed as described herein, at least one electrode 12, 12A (which may be a gel electrode including a gel comprising SHH as described herein), and a tube 14 containing conductive electrode gel (which gel may include SHH and/or GTN) as described herein). As used herein, electrode gel includes creams and ointments and other suitable vehicles.

For treating and/or regenerating lung utilizing Shh, see, e.g., Sriperumbudur et al., “Hedgehog: the key to maintaining adult lung repair and regeneration”, J Cell Commun Signal, 2017 March; 11(1): 95-96, published online 2016 Dec. 12. doi: 10.1007/s12079-016-0365-3 and University of Pennsylvania School of Medicine “Pinpointing gene that regulates repair, regeneration in adult lungs: New role for hedgehog gene offers better understanding of lung disease” ScienceDaily, 5 Oct. 2015, the contents of each of which are incorporated herein by this reference.

For treating and/or regenerating muscle utilizing Shh, see, e.g., Straface et al., “Sonic hedgehog regulates angiogenesis and myogenesis during post-natal skeletal muscle regeneration” J Cell Mol Med. 2009 August; 13(8b): 2424-2435, the contents of which are incorporated herein by this reference.

For treating and/or regenerating vascular smooth muscle cells utilizing Shh, see, e.g., Li et al., “Sonic Hedgehog Signaling Induces Vascular Smooth Muscle Cell Proliferation via Induction of the G1 Cyclin-Retinoblastoma Axis” Arteriosclerosis, Thrombosis, and Vascular Biology 2010; 30(9):1787-1794 (September 2010), the contents of which are incorporated herein by this reference.

For treating and/or regenerating the adrenal gland utilizing Shh, see, e.g., Finco et al., “Sonic Hedgehog and WNT Signaling Promote Adrenal Gland Regeneration in Male Mice” Endocrinology, Vol. 159, Issue 2, February 2018, pp 579-596, doi.org/10.1210/en.2017-03061, the contents of which are incorporated herein by this reference.

In certain embodiments, e.g., in the treatment of a subject suffering from cancer, down regulation of the expression and/or release of Sonic hedgehog may be of benefit. See, e.g., Ma et al. “Downregulation of Wnt signaling by sonic hedgehog activation promotes repopulation of human tumor cell lines”, Disease Models & Mechanisms 2015 8: 385-391; doi: 10.1242/dmm.018887, the contents of which are incorporated herein by this reference.

The bioelectric stimulator may also modulate the expression (e.g., upregulate expression of SDF-1, IGF-1, PDGF, follistatin, and tropoelastin).

Sonic hedgehog is as described above. Follistatin promotes muscle growth and counteracts myostatin. SDF-1 is generally for recruiting stem cells and maturing blood vessels. IGF-1 is for DNA repair. PDGF is a second stem cell homing factor and helps tissue regeneration. Any one of the protein expression signals work well on their own for organ regeneration, but they work better together. SDF-1 is a powerful regeneration protein, as is IGF-1.

Various bioelectric signals for modulating proteins are disclosed in US 20180064935 to Leonhardt et al. (Mar. 8, 2018), the contents of which are incorporated herein by this reference.

The pacing infusion lead may be constructed or purchased from the same suppliers that build standard heart pacemaker leads. Pacing infusion leads may be purchased from a variety of OEM vendors. The pacing infusion lead may, for example, be a standard one currently used in heart failure pacing studies in combination with drug delivery.

An infusion and electrode wide area patch may be constructed by cutting conduction polymer to a desired shape, and forming plastic into a flat bag with outlet ports in strategic locations.

Micro stimulators may be purchased or constructed in the same manner heart pacemakers have been made since the 1960's. When used with a micro infusion pump, such pumps can be purchased or produced similar to how they have been produced for drug, insulin, and pain medication delivery since the 1970's. The programming computer can be standard laptop computer. The programming wand customary to wireless programming wands may be used to program heart pacers.

Both wireless non-invasive and/or implantable wire lead (“electrode”) based means may be used to deliver the regeneration and healing promoting bioelectric signals to target organs.

A wireless, single lumen infusion pacing lead or infusion conduction wide array patch may all be used to deliver the regeneration signals and substances to the organ of interest to be treated or they may be used in combination.

A re-charging wand for use herein is preferably similar to the pacemaker re-charging wand developed by Alfred Mann in the early 1970's for recharging externally implantable pacemakers.

Bioelectric stimulation can be done with the described microstimulator, which can have a pacing infusion lead with, e.g., a corkscrew lead placed/attached at, e.g., the center of the tissue to be stimulated and/or treated.

The microstimulator is actuated and runs through programmed signals to signal the release of, e.g., Sonic hedgehog. In such a method, the electrical signal may be measured three (3) mm deep into the tissue.

Relationship Between the Components:

The micro voltage signal generator is attached to the pacing infusion lead with, e.g., a corkscrew tip, deep vein stimulation lead (Medtronic) (e.g., for bioelectric stimulation of the brain), or conductive polymer bandage or patch to the tissue or organ to be treated. An external signal programmer may be used to program the micro voltage signal generator with the proper signals for treatment including the Sonic hedgehog producing signal. The device battery may be re-chargeable with an external battery charging wand.

The essential elements are the micro voltage signal generator and the means for delivering the signal to the target tissue.

The signal generator may be external or internal. The transmission of the signal may be wireless, via liquid and/or via wires.

The tissue contact interface may be, e.g., a patch or bandage or may be via electrodes or leads. FDA cleared gel tape electrodes (Mettler) may be used for skin delivery. Electro acupuncture needles may be used to ensure the signals positively reach target tissues under the skin.

The invention is further described by the following illustrative Examples.

### EXAMPLES

### Example I

Electrode Gel: 100 ml of aloe vera gel is placed into a plastic resealable container. One tablespoon of salt (NaCl) is added to the gel, and the mixture is stirred until the salt dissolves completely.

A different electrode gel (e.g., TAC-Gel Electro Conductive & Adhesive Gel) may alternatively be used. Many such gels are in the form of a hydrogel that is efficient in (1) binding the electrode to the patient's skin and (2) efficiently dispersing electrical stimulation to the target area(s).

To this mixture is added the amount of recombinant human SHH to meet the desired concentration of SHH (e.g., 0.1% to 66% by weight), which is mixed thoroughly into the mixture. The recombinant human SHH may be purchased (e.g., from Acro Biosystems of Newark, Del., US) and/or recombinantly produced and purified as described in, e.g., Yu. et al. “Hair growth-promoting effect of recombinant human sonic hedgehog proteins.” Biomed Dermatol 3, 7 (2019); doi.org/10.1186/s41702-019-0047-x.

A permeation enhancer may be incorporated into the mixture to enhance delivery of the SHH.

### Example II

Studies were performed on osteoblasts, focusing on the expression of Sonic hedgehog (SHH). The results as are follows.

SHH had a 2.19 E+05 mRNA level/Glyceraldehyde-3-phosphate dehydrogenase (GAPDH—a default reference gene in quantitative mRNA profiling) fold gene upregulation at a frequency/pulse width duration of 5 Hz/1 ms.

A Table of results on osteoblasts at different frequencies follows:

Fold Gene Expression

| Sample | SHH |
|---|---|
| 5 Hz/1 ms | 3.17E+05 |
| 10 Hz/1 ms | 1.27E+05 |
| 20 Hz/1 ms | 1.37E+05 |
| 50 Hz/1 ms | 1.53E+05 |
| 100 Hz/1 ms | 8.47E+04 |

### Example III

Method of treating ED. A subject is treated for erectile dysfunction (ED) by applying an electrode to tissue at or near the subject's groin area so as to provide a bioelectric signal that increases the expression of Sonic hedgehog (SHH) in the tissue. For example, such a bioelectric signal, as measured at the cellular level of the tissue, comprises a biphasic pulse with/at a frequency/amplitude range of 40 Hz to 100 Hz at greater than or equal to 100 μA, or a biphasic pulse at from 5 Hz to 20 Hz at greater than or equal to 100 μA.

The electrode is a gel electrode. The gel electrode comprises a gel comprising 30% (w/w) SHH. The gel electrode gel may further comprise glyceryl trinitrate.

Before application of the electrode to the groin area, an electrode gel is applied to the electrode or to the groin area, wherein the electrode gel comprises SHH.

The electrode gel may further comprise glyceryl trinitrate.

The voltage and the current are increased until the subject feels a slight tingling at the stimulation area.

### REFERENCES (THE CONTENTS OF THE ENTIRETY OF EACH OF WHICH IS INCORPORATED HEREIN BY THIS REFERENCE)

Angeloni et al. “Regeneration of the cavernous nerve by sonic hedgehog using aligned peptide amphiphile nanofibers.” Biomaterials. 2011; 32:1091-1101.
Bond et al. “Peptide amphiphile nanofiber delivery of sonic hedgehog protein to reduce smooth muscle apoptosis in the penis after cavernous nerve resection.” J Sex Med. 2011 January; 8(1):78-89. doi: 10.1111/j.1743-6109.2010.02001.x. Epub 2010 Aug. 30. PMID: 20807324; PMCID: PMC3108851.
Choe et al. “Optimization of Sonic Hedgehog Delivery to the Penis from Self-Assembling Nanofiber Hydrogels to Preserve Penile Morphology after Cavernous Nerve Injury.” Nanomedicine. 2019 August; 20: 102033. doi: 10.1016/j.nano.2019.102033. Epub 2019 Jun. 5. PMID: 31173931; PMCID: PMC7194284.
Choe et al. “Sonic hedgehog delivery from self-assembled nanofiber hydrogels reduces the fibrotic response in models of erectile dysfunction.” Acta Biomater. 2016 Mar. 1; 32:89-99. doi: 10.1016/j.actbio.2016.01.014. Epub 2016 Jan. 14. PMID: 26776147; PMCID: PMC4831732.
Columbia, “Implant Procedure Concepts—Pacemaker, ICD and CRT Overview,” columbia.edu/itc/hs/medical/hickey/docs/Pacemaker,%20ICD%20and%20CRT%200verview%20022007.pdf.
Davis and Reisman “Development of a novel topical formulation of glyceryl trinitrate for the treatment of erectile dysfunction.” Int J Impot Res 32, 569-577 (2020); doi.org/10.1038/s41443-019-0227-7.
Dobbs et al. “Sonic hedgehog regulation of cavernous nerve regeneration and neurite formation in aged pelvic plexus.” Exp Neurol. 2019 February; 312:10-19. doi: 10.1016/j.expneurol.2018.11.001. Epub 2018 Nov. 2. PMID: 30391523; PMCID: PMC6342483.
Dobbs et al. “Peptide amphiphile delivery of sonic hedgehog protein promotes neurite formation in penile projecting neurons.” Nanomedicine. 2018 October; 14(7):2087-2094. doi: 10.1016/j.nano.2018.06.006. Epub 2018 Jul. 4. PMID: 30037776; PMCID: PMC6241323.
Dobbs et al., “019 Sonic Hedgehog Promotes Cavernous Nerve Regeneration by Inducing Cavernous Nerve Sprouting and Sprouting Potential is Reduced with Age,” J. Sex. Med. February 2018, Volume 15, Issue 2, Supplement 1, Page S10.
Fu et al. “Sonic hedgehog regulates the proliferation, differentiation, and migration of enteric neural crest cells in gut” J Cell Biol (2004) 166 (5): 673-684; doi.org/10.1083/jcb.200401077.
Hopkins Medicine, “Overview of Pacemakers and Implantable Cardioverter Defibrillators(ICDs).”
Hu et al. “A systematic summary of survival and death signalling during the life of hair follicle stem cells.” Stem Cell Res Ther. 2021 Aug. 11; 12(1):453. doi: 10.1186/s13287-021-02527-y. PMID: 34380571; PMCID: PMC8359037.
Malave, L., Zuelke, D. R., Uribe-Cano, S. et al. “Dopaminergic co-transmission with sonic hedgehog inhibits abnormal involuntary movements in models of Parkinson's disease and L-Dopa induced dyskinesia.” Commun Biol 4, 1071 (August 2021); doi.org/10.1038/s42003-021-02567-3.
Marrachelli et al. “Sonic hedgehog carried by microparticles corrects angiotensin II-induced hypertension and endothelial dysfunction in mice.” PLoS One. 2013 Aug. 16; 8(8):e72861. doi: 10.1371/journal.pone.0072861. PMID: 23977364; PMCID: PMC3745429.
Martin et al. “Peptide amphiphile nanofiber hydrogel delivery of Sonic hedgehog protein to the penis and cavernous nerve suppresses intrinsic and extrinsic apoptotic signaling mechanisms, which are an underlying cause of erectile dysfunction.” Nanomedicine. 2021 October; 37:102444. doi: 10.1016/j.nano.2021.102444. Epub 2021 Jul. 24. PMID: 34314869; PMCID: PMC8464506.
Ochoa et al., “Hedgehog signaling is critical for normal liver regeneration after partial hepatectomy in mice,” Hepatology. 2010 May; 51(5): 1712-1723. doi: 10.1002/hep.23525.
M. Paul “Protein Could Heal Erectile Dysfunction after Surgery” Northwestern Now (Jun. 8, 2010); northwestern.edu/newscenter/stories/2010/06/sonic.html.
Park et al. “Sonic hedgehog intradermal gene therapy using a biodegradable poly(β-amino esters) nanoparticle to enhance wound healing” Biomaterials 33(35):9148-9156 (2012); doi.org/10.1016/j.biomaterials.2012.09.005.
Podlasek C A. “Sonic hedgehog, apoptosis, and the penis.” J Sex Med. 2009 March; 6 Suppl 3(Suppl 3):334-9. doi: 10.1111/j.1743-6109.2008.01192.x. PMID: 19267857; PMCID: PMC2720523.
Podlasek et al. “Sonic hedgehog, the penis and erectile dysfunction: a review of sonic hedgehog signaling in the penis.” Curr Pharm Des. 2005; 11(31):4011-27. doi: 10.2174/138161205774913408. PMID: 16378507.
Prochazka et al., “Cocktail of Factors from Fat-derived Stem Cells Shows Promise for Critical Limb Ischemia,” www.sciencenewsline.com/news/2016012204520017.html (Jan. 21, 2016).
Salcedo et al., “Low current electrical stimulation upregulates cytokine expression in the anal sphincter,” Int. J. Colorectal Dis., 2012 February; 27(2):221-5. doi: 10.1007/s00384-011-1324-3. Epub (October 2011).
Scholefield et al. “A dose finding study with 0.1%, 0.2%, and 0.4% glyceryl trinitrate ointment in patients with chronic anal fissures.” Gut vol. 52, 2 (2003): 264-9. doi:10.1136/gut.52.2.264.
Su el al. “High frequency stimulation induces sonic hedgehog release from ocampal neurons.” Sci Rep 7, 43865 (2017) doi.org/10.1038/srep43865.
Yamada et al. “The Sonic Hedgehog signaling pathway regulates inferior alveolar nerve regeneration” Neuroscience Letters Volume 671, (3 Apr. 2018), pages 114-119.
Yu. et al. “Hair growth-promoting effect of recombinant human sonic hedgehog proteins.” Biomed Dermatol 3, 7 (2019); doi.org/10.1186/s41702-019-0047-x.
Zhang et al. “Role of VIP and Sonic Hedgehog Signaling Pathways in Mediating Epithelial Wound Healing, Sensory Nerve Regeneration, and Their Defects in Diabetic Corneas” Diabetes 2020 July; 69(7): 1549-1561; doi.org/10.2337/db19-0870.
Zhou, Dong et al. “Sonic hedgehog signaling in kidney fibrosis: a master communicator.” Science China. Life sciences vol. 59, 9 (2016): 920-9. doi:10.1007/s11427-016-0020-y.
U.S. Pat. No. 10,960,206 to Leonhardt et al.
U.S. Pat. No. 10,342,968 to Hasui et al. (Jul. 9, 2019) for “Electrode pad used for iontophoresis treatment”.

What is claimed is:

1. A method of treating a subject for erectile dysfunction (ED), the method comprising:
applying an electrode to tissue at or near the subject's groin area so as to provide a bioelectric signal that increases the expression of Sonic hedgehog (SHH) in the tissue,
wherein the bioelectric signal as measured at the cellular level of the tissue comprises:
a biphasic pulse with/at a frequency/amplitude range of 40 Hz to 100 Hz at greater than or equal to 100 μA, or
a biphasic pulse at from 5 Hz to 20 Hz at greater than or equal to 100 μA.

2. The method of claim 1, wherein the electrode is a gel electrode.

3. The method according to claim 2, wherein the gel electrode comprises a gel comprising SHH.

4. The method according to claim 3, wherein the gel electrode gel further comprises glyceryl trinitrate.

5. The method according to claim 1, further comprising:
applying an electrode gel to the electrode or to the groin area before application of the electrode to the groin area, wherein the electrode gel comprises SHH.

6. The method according to claim 5, wherein the electrode gel further comprises glyceryl trinitrate.

7. The method according to claim 1, further comprising:
applying an electrode gel to the electrode or to the groin area before application of the electrode to the groin area, wherein the electrode gel comprises glyceryl trinitrate.

8. The method according to claim 1, wherein the bioelectric signal has a frequency/pulse width duration of 5 Hz/1 ms.

9. The method according to claim 1, wherein the bioelectric signal has a frequency/pulse width duration of 10 Hz/1 ms.

10. The method according to claim 1, wherein the bioelectric signal has a frequency/pulse width duration of 20 Hz/1 ms.

11. The method according to claim 1, wherein the bioelectric signal has a frequency/pulse width duration of 50 Hz/1 ms.

12. The method according to claim 1, wherein the bioelectric signal has a frequency/pulse width duration of 40 Hz/1 ms.

13. The method according to claim 1, wherein the bioelectric signal has a frequency/pulse width duration of 100 Hz/1 ms.

14. A gel electrode for conducting a bioelectric signal to a subject, wherein the improvement comprises:
incorporating into a gel contained within the gel electrode Sonic hedgehog, glycerol trinitrate, or a combination thereof.

15. An electrode gel for conducting a bioelectric signal to a subject, wherein the improvement comprises:
incorporating into the electrode gel Sonic hedgehog, glycerol trinitrate, or a combination thereof.
